(19) United States
(12) Patent Application Publication
(10) Pub. No.: US 2026/0204395 A1
(43) Pub. Date: Jul. 16, 2026

(54) INFORMATION PROCESSING APPARATUS, RADIATION IMAGING SYSTEM, INFORMATION PROCESSING METHOD, AND NON TRANSITORY COMPUTER-READABLE STORAGE MEDIUM

(71) Applicant: CANON KABUSHIKI KAISHA, Tokyo (JP)

(72) Inventors: DAISUKE YAMADA, Kanagawa (JP); MANAMI HIKOSAKA, Kanagawa (JP); KAZUMA OBARA, Tokyo (JP); MASAYA KAWAI, Kanagawa (JP); IORI YOSHIDA, Tokyo (JP)

(21) Appl. No.: 19/415,144

(22) Filed: Dec. 10, 2025

(30) Foreign Application Priority Data
Jan. 7, 2025 (JP) 2025-002343

(51) Int. Cl. G16H 30/20; G06F 3/04842; G06T 7/00

(52) U.S. Cl. CPC G16H 30/20; G06F 3/04842; G06T 7/0014; G06T 2200/24; G06T 2207/30008

(57) ABSTRACT

An information processing apparatus, which processes an image obtained by radiation imaging, comprises a display control unit configured to cause a display to display a protocol selection portion that displays first imaging for bone mineral density measurement and second imaging different from the first imaging for the bone mineral density measurement in an identifiable and selectable manner before the radiation imaging.

### BACKGROUND

### Field of the Technology

The present disclosure relates to an information processing apparatus, a radiation imaging system, an information processing method, and a non transitory computer-readable storage medium.

### Description of the Related Art

A measurement method called a bone mineral density (BMD) method has been used for diagnosis or the like on osteoporosis. As a method of measuring a bone mineral quantity in the bone, a dual-energy X-ray absorptiometry (DXA: also called a DXA method) is known, which is a technique of measuring a bone mineral density from the difference in X-ray absorption coefficient between a soft tissue and a bone tissue by using two types of X-rays with different energy distributions.

As an apparatus dedicated to bone mineral density measurement using the DXA method, there is available, for example, an apparatus that includes a line sensor and is configured to alternately apply high-energy X-rays and low-energy X-rays at regular intervals. In addition, there is available a filter type apparatus that has a filter placed in front of an X-ray tube to change X-rays into different irradiation energies.

Recently, with the widespread use of digital image diagnosis using radiation images obtained by a general imaging apparatus, the corresponding technique has begun to be applied to bone mineral density measurement. In a case where bone mineral density imaging is performed by using a radiation detection apparatus for general imaging (flat panel detector to be also referred to as an FPDe hereinafter), an image is obtained by irradiating the entire sensor surface with X-rays (cone beam imaging). Accordingly, the time taken for one imaging cycle is short, and body movement is suppressed, thus reducing the physical strain on a patient. The radiation detection apparatus for general imaging (FPDe) can obtain images with higher resolution and higher sensitivity than the conventional apparatus dedicated to bone mineral density measurement and hence allows the use of bone mineral density measurement images for diagnosis.

With the above imaging apparatus that can perform both general imaging and bone mineral density measurement imaging for measuring a bone mineral density, the user may perform imaging based on wrong imaging conditions, for example, imaging based on general imaging conditions in spite of using examination information for bone mineral density measurement. Accordingly, Japanese Patent Laid-Open No. 2023-183893 proposes an arrangement that prevents bone mineral density measurement under imaging conditions unsuitable for bone mineral density measurement and prevents unnecessary exposure on a subject.

Although the arrangement disclosed in patent literature 1 needs to obtain imaging conditions before imaging, the imaging conditions include no information about the radiation generating apparatus and instruments attached to the FPDe. In this case, the instruments include, for example, a stop that is attached to the radiation generating apparatus to limit or fix an irradiation field and a grid that is attached to the FPDe to reduce the influence of scattered radiation. In addition, it is assumed that general imaging and bone mineral density measurement respectively require different instruments. Accordingly, it is necessary to determine whether such instruments are attached and appropriate instruments are attached (for example, the stop size and the grid direction and interval are appropriate). It is difficult to obtain such information before imaging.

Accordingly, the imaging apparatus that can perform both general imaging and bone mineral density measurement may perform bone mineral density measurement using images captured under inappropriate imaging conditions upon failing to change an instrument such as a stop or grid, thus failing to perform bone mineral density measurement based on a correct measurement result. For this reason, the imaging apparatus that can perform both general imaging and bone mineral density measurement imaging for measuring a bone mineral density is required to perform imaging with appropriate settings upon accurately identifying first imaging for bone mineral density measurement and general imaging (to be also referred to as second imaging hereinafter) different from the first imaging for bone mineral density measurement without mixing up with each other.

### SUMMARY

The present disclosure has been made in consideration of the above problem and provides a technique that can reduce the work burden on the user and improve the reliability of diagnosis by improving the efficiency of checking first imaging for bone mineral density measurement and second imaging different from the first imaging for bone mineral density measurement, facilitating the checking, and preventing misuse.

According to one aspect of the present disclosure, there is provided an information processing apparatus that processes an image obtained by radiation imaging, the apparatus comprising a display control unit configured to cause a display to display a protocol selection portion that displays first imaging for bone mineral density measurement and second imaging different from the first imaging for the bone mineral density measurement in an identifiable and selectable manner before the radiation imaging.

Further features of the present disclosure will become apparent from the following description of exemplary embodiments (with reference to the attached drawings).

### BRIEF DESCRIPTION OF THE DRAWINGS

FIG. 1 is a block diagram showing an arrangement example of a radiation imaging system according to an embodiment of the present disclosure;

FIG. 2 is a flowchart showing an example of imaging processing and output processing according to the first embodiment;

FIG. 3 is a view showing a GUI screen display example before imaging according to the first embodiment;

FIG. 4 is a view showing a GUI screen display example after general imaging and before bone mineral density measurement imaging according to the first embodiment;

FIG. 5 is a view showing a GUI screen display example after bone mineral density measurement imaging according to the first embodiment; and

FIG. 6 is a view showing a GUI screen display example before imaging according to the second embodiment.

### DESCRIPTION OF THE EMBODIMENTS

Hereinafter, embodiments will be described in detail with reference to the attached drawings. Note, the following embodiments are not intended to limit the scope of the claims. Multiple features are described in the embodiments, but it is not the case that all such features are required, and multiple such features may be combined as appropriate. Furthermore, in the attached drawings, the same reference numerals are given to the same or similar configurations, and redundant description thereof is omitted.

Note that radiation in the disclosed technique can include not only α-rays, β-rays, and γ-rays that are beams generated by particles (including photons) emitted by radioactive decay but also beams having energy equal to or higher than the energy of these beams, for example, X-rays, particle rays, and cosmic rays.

### First Embodiment

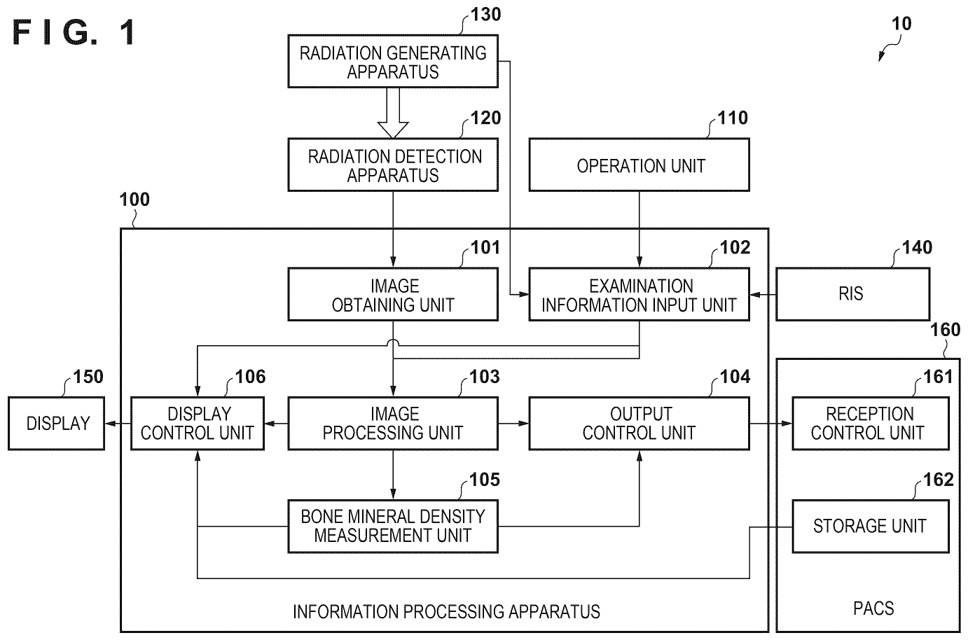

FIG. 1 is a block diagram showing an arrangement example of a bone mineral density measurement system 10 (to be also referred to as a radiation imaging system hereinafter) according to the first embodiment. An information processing apparatus 100 is an apparatus for processing an obtained radiation image. The information processing apparatus 100 is connected to a radiology information system (RIS) 140 and a picture archiving and communication systems (PACS) 160 and controls radiation imaging using a radiation detection apparatus 120 and a radiation generating apparatus 130. The RIS 140 transmits examination information to the information processing apparatus 100. The PACS 160 is an image server that manages the images processed by the information processing apparatus 100. Note that the RIS 140 and the PACS 160 connected to the information processing apparatus 100 may be incorporated in the same PC or may be respectively incorporated in different PCs and connected to each other via a network.

The radiation detection apparatus 120 detects the radiation emitted from the radiation generating apparatus 130 and having passed through a subject (not shown) and outputs image data corresponding to the radiation. The DXA method is a method of measuring a bone mineral density based on the difference in radiation absorption coefficient between a soft tissue and a bone tissue from a plurality of images obtained by applying two types of radiation with different energy distributions from the radiation generating apparatus 130. A radiation detection apparatus may be used, which can obtain radiation images by detecting radiation with different energies upon one irradiation with radiation. Note that image data can be reworded as, for example, an image, medical image, or radiation image.

More specifically, the radiation detection apparatus 120 detects the radiation transmitted through the subject as electric charge corresponding to the penetrating radiation dose. For example, an apparatus used as the radiation detection apparatus 120 is a direct conversion type sensor that directly converts radiation into electric charge, such as an a-Se sensor that converts radiation into electric charge, or an indirect conversion type sensor using a scintillator such as a CsI scintillator that converts radiation into visible light and a photoelectric conversion element such as an a-Si element. In addition, the radiation detection apparatus 120 generates image data by A/D-converting detected electric charge and outputs the data to the information processing apparatus 100.

The information processing apparatus 100 is connected to the radiation detection apparatus 120 via, for example, a wired or wireless network or dedicated line. The radiation detection apparatus 120 detects the radiation generated by the radiation generating apparatus 130 and outputs the image data generated based on the detected radiation to the information processing apparatus 100. The information processing apparatus 100 generates an image by performing image processing on the image data output from the radiation detection apparatus 120 and displays the image on a display 150. The display 150 is implemented by, for example, a liquid crystal display or the like and displays various types of information to the operator (for example, an imaging technician or doctor). An operation unit 110 is constituted by, for example, a mouse and operation buttons and accepts various types of instructions from the operator. Note that the display 150 and the operation unit 110 may be implemented as a touch panel integrating them.

The information processing apparatus 100 also has a function of controlling each constituent element. The information processing apparatus 100 outputs an image to the display 150 and provides a graphical user interface (GUI) using the display 150 while controlling the operation of the radiation detection apparatus 120.

The information processing apparatus 100 notifies the timing when the radiation generating apparatus 130 generates radiation and imaging conditions for radiation to be applied. The information processing apparatus 100 notifies the radiation generating apparatus 130 of the timing of generating radiation by transmitting an irradiation enable signal to the radiation generating apparatus 130. An image obtaining unit 101 of the information processing apparatus 100 controls the timing of capturing image data in the radiation detection apparatus 120 and the timing of obtaining image data from the radiation detection apparatus 120.

An image processing unit 103 of the information processing apparatus 100 performs image processing such as noise removal and tone processing on the image data obtained from the radiation detection apparatus 120. The image processing unit 103 can also perform image processing such as trimming or rotation on the image output from the radiation detection apparatus 120.

An examination information input unit 102 of the information processing apparatus 100 accepts an instruction from the operator and inputs examination information. As a method of inputting examination information, the examination information input unit 102 may input the examination information received from the RIS 140, or the operator may manually input the examination information via the operation unit 110. The examination information input to the examination information input unit 102 is managed in association with the image data captured by the radiation detection apparatus 120.

An output control unit 104 of the information processing apparatus 100 converts the image output from the image processing unit 103 into the digital imaging and communications in medicine (DICOM) form and outputs it to the PACS 160. The output control unit 104 controls the output of images obtained by general imaging and a plurality of images with different energy distributions obtained by bone mineral density measurement imaging. A reception control unit 161 of the PACS 160 receives the image output from the output control unit 104 of the information processing apparatus 100 and stores the received image in a storage unit 162.

A display control unit 106 of the information processing apparatus 100 controls the display of the display 150. The display control unit 106 can perform display control to display various types of images processed by each unit in the information processing apparatus 100. The display control unit 106 can also perform display control to cause the display 150 to display image data stored in the storage unit 162 of the PACS 160 as an image used for past examination.

The functional arrangement of each of the image obtaining unit 101, the examination information input unit 102, the image processing unit 103, the output control unit 104, a bone mineral density measurement unit 105, and the display control unit 106 in the information processing apparatus 100 may be implemented by causing the central processing unit (CPU) of the information processing apparatus 100 or a dedicated or general-purpose processor to execute a program. Alternatively, each functional arrangement may be implemented by hardware such as a field programmable gate array (FPGA) or application specific integrated circuit (ASIC). The information processing apparatus 100 can also implement various types of functions by combining software processing using a processor and programs and hardware processing.

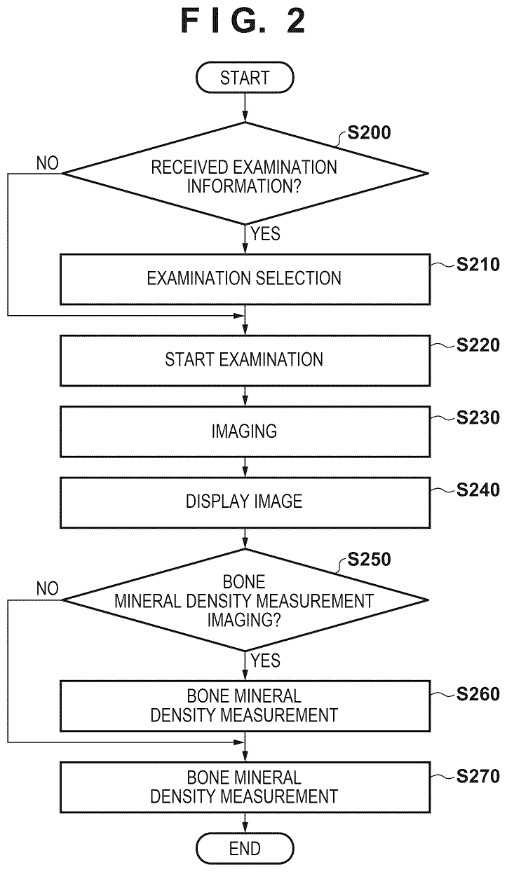

Imaging to capture a radiation image and output processing according to the present embodiment will be described next in accordance with the flowchart of FIG. 2.

### (S200: Examination Information Input)

In step S200, the examination information input unit 102 accepts an instruction from the operator and inputs examination information. As a method of inputting examination information, the operator can select whether to use examination information received from the RIS 140 or manually input examination information. If the operator selects the method of accepting examination information from the RIS 140 (S200/YES), the process shifts to step S210. In contrast to this, if the operator selects the method of manually inputting examination information in step S200 (S200/NO), the examination information input unit 102 accepts the selection of manual input of examination information from the operator, and the process then shifts to step S220.

### (S210: Examination Selection)

In step S210, the examination information input unit 102 sets one piece of the examination information received from the RIS 140 as an examination target. The examination information received from the RIS 140 may be displayed on the display 150. In the processing of setting an examination target, for example, one piece of a plurality of pieces of examination information displayed in a list form on the display 150 is set as an imaging target in accordance with the operation input selected by the operator.

### (S220: Start of Examination)

In step S220, the examination information is confirmed by an operation input from the operator, and the information processing apparatus 100 transmits a signal for shifting to a preparation state to the radiation detection apparatus 120 associated with imaging of the examination information. In accordance with the signal transmitted from the information processing apparatus 100, if no bias voltage is applied to a two-dimensional imaging element, the radiation detection apparatus 120 controls a bias power supply using a main control circuit to apply a bias voltage to the two-dimensional imaging element. Thereafter, in order to read out dark current signals accumulated in pixels, a driving circuit performs initialization to read out image signals from a pixel array. After the initialization, the radiation detection apparatus 120 transmits, to the information processing apparatus 100, state information indicating a state in which the preparation for obtaining a radiation image is completed.

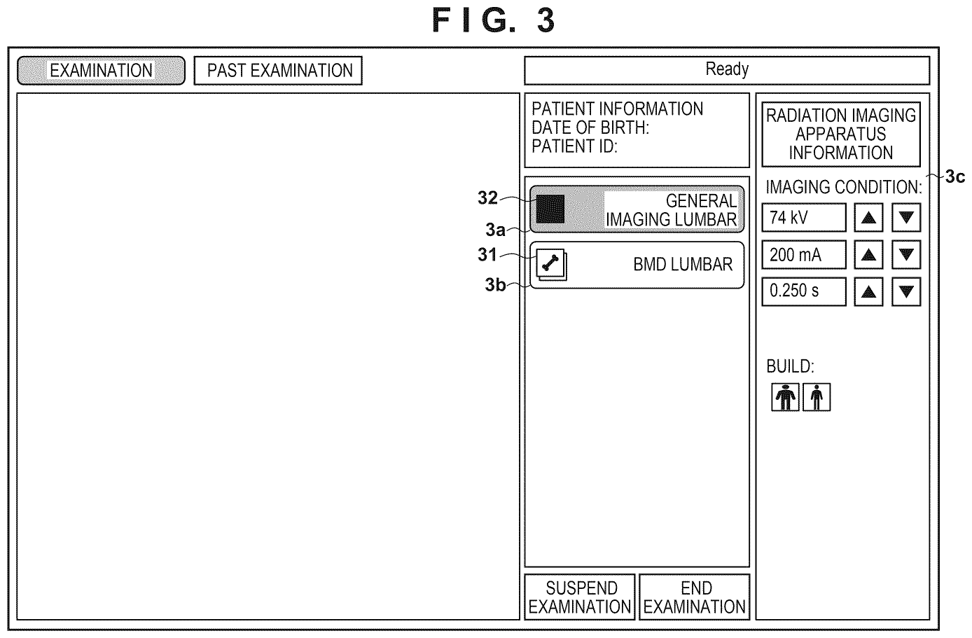

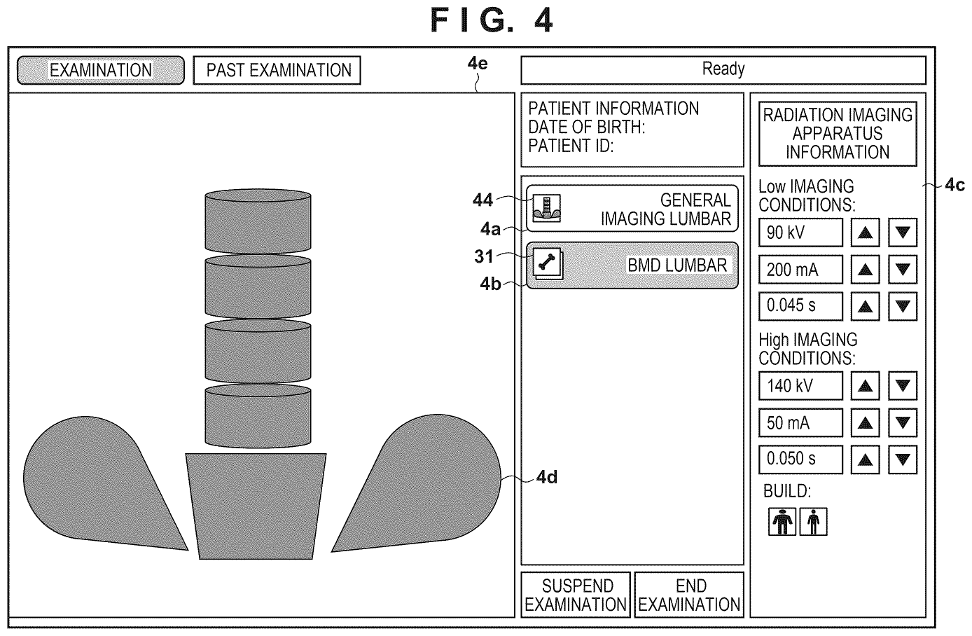

The display control unit 106 controls display of a graphical user interface to be displayed on the display 150. The GUI screen examples (FIGS. 3 to 6) to be described below are displayed under the control of the display control unit 106. FIG. 3 shows a screen example (GUI screen example) of the graphical user interface displayed on the display 150. FIG. 3 shows a state before general imaging and bone mineral density measurement imaging with respect to the lumbar region. In the following description, in order to discriminate general imaging from bone mineral density measurement imaging, general imaging will also be referred to as imaging different from bone mineral density measurement imaging. The GUI screen example displays protocol selection portions for the selection of imaging protocols in a form that allows the user to identify bone mineral density measurement imaging and imaging (general imaging) different from bone mineral density measurement imaging before imaging. This screen example displays a protocol selection portion 3a that allows the user to identify general imaging and a protocol selection portion 3b that allows the user to identify bone mineral density measurement imaging. In the protocol selection portion 3a for general imaging, a thumbnail image display area 32 evoking the performing of general imaging to capture one image is displayed in the protocol selection portion 3a. In a case where general imaging is performed, a thumbnail image (44 in FIG. 4) obtained by reducing the image obtained by general imaging is displayed in the thumbnail image display area 32. In the protocol selection portion 3b, an icon 31 expressing bone mineral density measurement is displayed in the protocol selection portion 3b for bone mineral density measurement imaging to allow the user to identify the protocol selection portion 3b relative to the protocol selection portion 3a for general imaging. The protocol selection portions 3a and 3b function, for example, as buttons that allow selection in a touch panel form.

The GUI screen example also displays an imaging condition display section 3c that displays imaging conditions corresponding to a selected imaging protocol. For example, in a case where the protocol selection portion 3a is selected, imaging conditions for general imaging are displayed. In a case where the protocol selection portion 3b is selected, imaging conditions for bone mineral density measurement imaging are displayed.

The GUI screen example (FIG. 3) shows a state in which imaging conditions for general imaging are displayed while the protocol selection portion 3a is selected. If, for example, the protocol selection portion 3a is selected, the display color is changed (gray-out display in FIG. 3). The display colors of the protocol selection portions 3a and 3b enable the user to check whether general imaging or bone mineral density measurement imaging is selected.

The imaging condition display section 3c displays two types of irradiation conditions with different energies (High and Low) in the imaging conditions for bone mineral density measurement imaging (see, for example, FIG. 4), although the imaging conditions for general imaging include one type of radiation irradiation condition (kV, mA, s). The display of the imaging condition display section 3c enables the user to check whether general imaging or bone mineral density measurement imaging is selected. In a case where general imaging is performed under the imaging conditions displayed on the imaging condition display section 3c, as indicated by the GUI screen example shown in FIG. 4, the GUI screen displays a general captured image 4d as a radiation image (second radiation image) captured by second imaging.

### (S230: Imaging)

In step S230, the driving circuit of the radiation detection apparatus 120 detects the radiation applied by the radiation generating apparatus 130, reads out an obtained image signal by using a readout circuit, and generates a radiation image. Thereafter, the radiation detection apparatus 120 transmits the radiation image to the information processing apparatus 100. The image obtaining unit 101 of the information processing apparatus 100 obtains the radiation image transmitted from the radiation detection apparatus 120.

### (S240: Image Display)

In step S240, the image processing unit 103 obtains the examination information input at the start of imaging from the examination information input unit 102. The examination information may be deployed in a memory or held in a storage device such as a database or file (not shown). In step S240, the image processing unit 103 may read out the examination information from the storage device. In this case, the examination information indicates, for example, the examination information input in step S200 or S210. For example, the examination information includes an imaging region subjected to radiation imaging, imaging technique information (for example, general imaging or bone mineral density measurement imaging), imaging conditions for each imaging technique (for example, the size of the stop attached to the radiation generating apparatus 130 and the direction and interval of the grid attached to the radiation detection apparatus 120).

FIG. 4 shows the GUI screen example displayed on the display 150 after general imaging. In the GUI screen example shown in FIG. 4, the general captured image 4d obtained after general imaging is displayed in a display area 4e of an image. The thumbnail image 44 of the general captured image 4d is displayed on a protocol selection portion 4a.

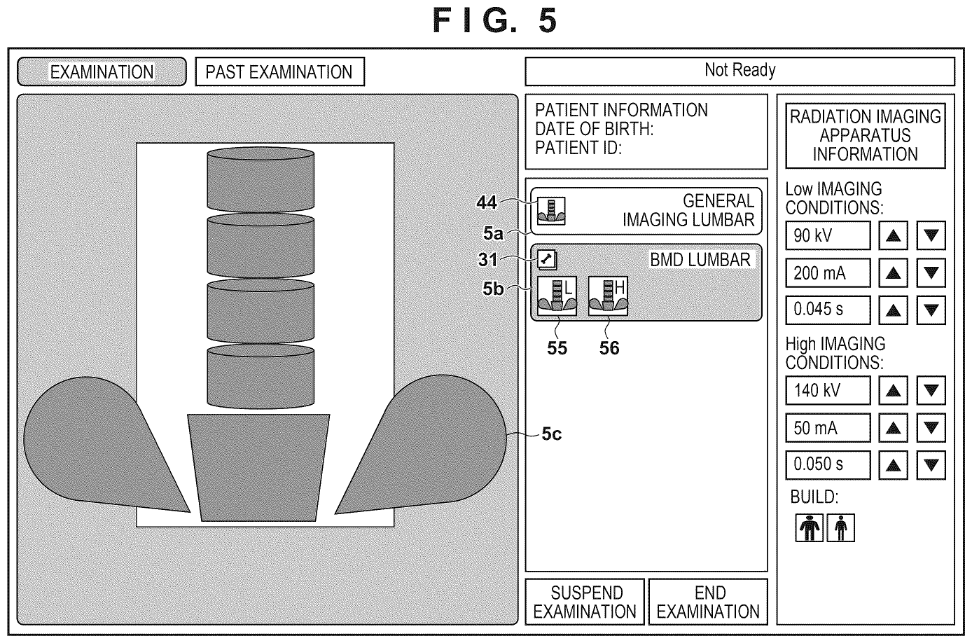

The GUI screen example shows a state in which a protocol selection portion 4b for bone mineral density measurement imaging is selected (gray-out display in FIG. 4). The GUI screen example (FIG. 4) also shows an imaging condition display section 4c for displaying imaging conditions corresponding to the selected imaging type (protocol). If the protocol selection portion 4b for bone mineral density measurement imaging is selected, imaging conditions for low-energy imaging for bone mineral density measurement and high-energy imaging are displayed on the imaging condition display section 4c. In this manner, the imaging condition display section (3c or 4c) displays imaging conditions corresponding to a selected imaging type (protocol). Switching the display between the imaging condition display sections (3c and 4c) at the time of general imaging and the time of bone mineral density measurement imaging to enable the user to identify general imaging and bone mineral density measurement imaging. In a case where bone mineral density measurement imaging is performed under the imaging conditions displayed on the imaging condition display section 4c, the GUI screen displays a bone mineral density measurement captured image 5c as a radiation image (first radiation image) captured by the first imaging, as indicated by the GUI screen example shown in FIG. 5.

FIG. 5 shows a GUI screen example displayed on the display 150 after bone mineral density measurement imaging. The screen example shows a protocol selection portion 5a displaying the thumbnail image 44 after general imaging.

The screen example shows a protocol selection portion 5b after bone mineral density measurement imaging. The protocol selection portion 5b displays a thumbnail image 55 of a low-energy image for bone mineral density measurement and a thumbnail image 56 of a high-energy image. The protocol selection portion 5b also displays the icon 31 expressing bone mineral density measurement. In addition, after bone mineral density measurement imaging, the icon 31 expressing bone mineral density measurement, the thumbnail image 55 of the low-energy image for bone mineral density measurement, and the thumbnail image 56 of the high-energy image are displayed on the protocol selection portion 5b. This enables the user to identify the protocols for general imaging and bone mineral density measurement imaging.

### (S250: Bone Mineral Density Measurement Determination)

In step S250, the bone mineral density measurement unit 105 determines whether the displayed image is an image obtained by bone mineral density measurement imaging. If the image is obtained by bone mineral density measurement imaging (S250/YES), the process shifts to step S260.

### (S260: Bone Mineral Density Measurement)

In step S260, the bone mineral density measurement unit 105 measures the bone mineral density of a subject by using the image captured by bone mineral density measurement imaging and creates a report concerning the measurement result. Note that a series of processing (steps S250 and S260) concerning bone mineral density measurement may be activated automatically or in accordance with an instruction from the operator after the end of image display processing (step S240). That is, in an automatic operation, in a case where bone mineral density measurement imaging is performed, it is possible to execute bone mineral density measurement without any operation after image display in step S240. In contrast to this, in an operation corresponding to an instruction from the operator, for example, it is determined in step S250 whether bone mineral density measurement imaging is performed after image display in step S240. If bone mineral density measurement imaging is to be performed (S250/YES), the display control unit 106 causes the display 150 to display a button for shifting to bone mineral density measurement. Thereafter, if the operator presses the button displayed on the display 150, the process shifts to step S260. In this case, the operator can execute bone mineral density measurement at an arbitrary timing. In addition, bone mineral density measurement may be executed after the end of examination.

### (S270: End of Examination/Output)

In step S270, the information processing apparatus 100 finishes the examination executed in accordance with an input operation by the operator. At the end timing of the examination, the output control unit 104 outputs the image captured at the examination and an examination report to the PACS 160 as an external device. Note that the operation timing of output processing may not coincide with the end timing of the examination and may coincide with, for example, the timing of re-outputting an image or report after the end of the examination.

According to the present embodiment, in the imaging apparatus that can perform both general imaging and bone mineral density measurement imaging, it is possible to reduce the work burden on the user and improve the reliability of diagnosis by improving the efficiency of checking general imaging and bone mineral density measurement imaging and facilitating the discrimination between them, and preventing misuse.

### Second Embodiment

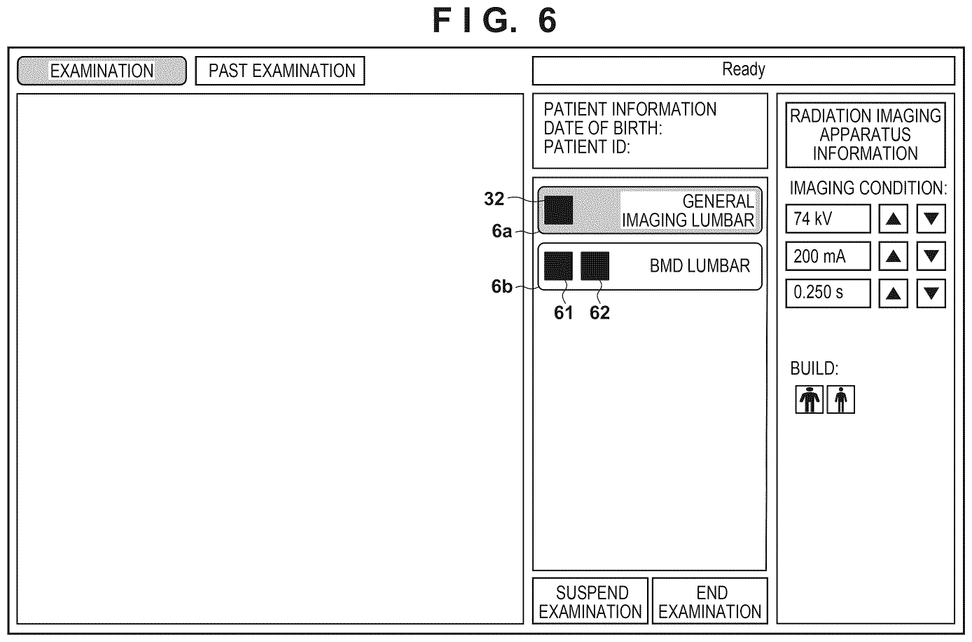

FIG. 6 shows a GUI screen example displayed on a display 150 before bone mineral density measurement imaging as an example of displaying protocols for general imaging and bone mineral density measurement imaging in an identifiable form.

The first embodiment is configured to display the icon 31 (FIG. 3) expressing bone mineral density measurement imaging configured to perform imaging to obtain two images with low energy and high energy. The GUI screen example shown in FIG. 6 displays thumbnail image display areas 61 and 62 evoking the performing of imaging to obtain two images with low energy and high energy on a protocol selection portion 6b for bone mineral density measurement. In a case where bone mineral density measurement imaging is performed, a thumbnail image of a low-energy image and a thumbnail image of a high-energy image are respectively displayed in the thumbnail image display areas 61 and 62. The number of radiation images obtained by imaging for bone mineral density measurement (first imaging) is different from the number of radiation images obtained by general imaging (second imaging) different from imaging (first imaging) for bone mineral density measurement. The number (for example, one) radiation images obtained by general imaging (second imaging) is smaller than the number (for example, two) of radiation images obtained by imaging (first imaging) for bone mineral density measurement performed by irradiation with two types of radiation with different energy distributions. In the second embodiment, a display control unit 106 causes the protocol selection portion 6b to display the thumbnail image display areas 61 and 62 that allow identification between the first imaging and the second imaging before radiation imaging as areas for displaying thumbnail images of radiation images obtained by the first imaging and the second imaging in different numbers.

In contrast, a protocol selection portion 6a for general imaging displays a thumbnail image display area 32 evoking the performing of general imaging to obtain one image. In a case where general imaging is performed, an image obtained by general imaging is displayed in the thumbnail image display area 32. The thumbnail image display areas 32, 61, and 62 displayed on the protocol selection portions 6a and 6b enable the user to easily discriminate and select general imaging or bone mineral density measurement imaging.

As indicated by the GUI screen example in FIG. 6, the display control unit 106 causes the protocol selection portion 6b to display the plurality of thumbnail image display areas 61 and 62 for the identification of the first imaging. As indicated by the GUI screen example in FIG. 5, the display control unit 106 causes a protocol selection portion (see, for example, 5b in FIG. 5) to display a plurality of thumbnail images 55 and 56 concerning the first imaging which are obtained after radiation imaging (bone mineral density measurement imaging) and the icon 31. The plurality of thumbnail images 55 and 56 concerning the first imaging are displayed on the protocol selection portion (5b in FIG. 5) while being inserted in the thumbnail image display areas 61 and 62.

As indicated by the GUI screen example shown in FIG. 6, the display control unit 106 causes the protocol selection portion 6a to display one thumbnail image display area 32 for the identification of the second imaging. As indicated by the GUI screen example shown in FIG. 5, the display control unit 106 causes a protocol selection portion (see, for example, 5a in FIG. 5) to display a thumbnail image 44 concerning the second imaging which is obtained after radiation imaging (general imaging). The thumbnail image 44 of an image concerning second imaging is displayed on a protocol selection portion (5a in FIG. 5) while being inserted in the thumbnail image display area 32.

According to the present embodiment, in the imaging apparatus that can perform both general imaging and bone mineral density measurement imaging, it is possible to reduce the work burden on the user and improve the reliability of diagnosis by improving the efficiency of checking general imaging and bone mineral density measurement imaging, facilitating the discrimination between them, and preventing misuse.

According to the present disclosure, it is possible to reduce the work burden on the user and improve the reliability of diagnosis by improving the efficiency of checking general imaging and bone mineral density measurement imaging, facilitating the discrimination between them, and preventing misuse.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2025-002343, filed Jan. 7, 2025, which is hereby incorporated by reference herein in its entirety.

What is claimed is:

1. An information processing apparatus that processes an image obtained by radiation imaging, the apparatus comprising a display control unit configured to cause a display to display a protocol selection portion that displays first imaging for bone mineral density measurement and second imaging different from the first imaging for the bone mineral density measurement in an identifiable and selectable manner before the radiation imaging.

2. The apparatus according to claim 1, wherein the display control unit causes the display to display an imaging condition display section that displays the first imaging and the second imaging in an identifiable manner before the radiation imaging in accordance with selection of the protocol selection portion.

3. The apparatus according to claim 2, wherein the display control unit causes the imaging condition display section to display a plurality of imaging conditions different in radiation energy in a case where the first imaging is selected by the protocol selection portion.

4. The apparatus according to claim 3, wherein the display control unit causes the imaging condition display section to display an imaging condition with first energy and an imaging condition with second energy higher than the first energy as the plurality of imaging conditions in a case where the first imaging is selected.

5. The apparatus according to claim 2, wherein the display control unit causes the imaging condition display section to display an imaging condition with one radiation energy in a case where the second imaging is selected by the protocol selection portion.

6. The apparatus according to claim 1, wherein the number of radiation images obtained by the first imaging is different from the number of radiation images obtained by the second imaging, and
the display control unit causes the protocol selection portion to display a thumbnail image display area that makes the first imaging and the second imaging identifiable before the radiation imaging as an area that displays thumbnail images of radiation images different in number obtained by the first imaging or the second imaging.

7. The apparatus according to claim 6, wherein the display control unit causes the protocol selection portion to display the plurality of thumbnail image display areas for identification of the first imaging.

8. The apparatus according to claim 7, wherein the display control unit causes the plurality of thumbnail image display areas to display thumbnail images of a plurality of images concerning the first imaging which are obtained after the radiation imaging.

9. The apparatus according to claim 6, wherein the display control unit causes the protocol selection portion to display one thumbnail image display area for identification of the second imaging.

10. The apparatus according to claim 9, wherein the display control unit causes the thumbnail image display area to display a thumbnail image of an image concerning the second imaging which is obtained after the radiation imaging.

11. The apparatus according to claim 1, wherein the display control unit causes the protocol selection portion to display an icon expressing the first imaging for the bone mineral density measurement so as to make the first imaging identifiable.

12. The apparatus according to claim 11, wherein the display control unit causes the protocol selection portion to display thumbnail images of a plurality images concerning the first imaging which are obtained after the radiation imaging and the icon.

13. The apparatus according to claim 1, wherein the display control unit causes the display to display the protocol selection portion corresponding to selected imaging of the first imaging and the second imaging upon changing a display color of the protocol selection portion.

14. The apparatus according to claim 6, wherein the number of radiation images obtained by the second imaging is smaller than the number of radiation images obtained by the first imaging.

15. An information processing apparatus that processes an image obtained by radiation imaging, the apparatus comprising a display control unit configured to cause a display to display a thumbnail image display area that makes identifiable first imaging for bone mineral density measurement and second imaging different from the first imaging for the bone mineral density measurement before the radiation imaging.

16. A radiation imaging system comprising:
a radiation detection apparatus configured to detect radiation; and
an information processing apparatus defined in claim 1 which is communicably connected to the radiation detection apparatus.

17. An information processing method for an information processing apparatus that processes an image obtained by radiation imaging, the method comprising causing a display to display a protocol selection portion that display first imaging for bone mineral density measurement and second imaging different from the first imaging for the bone mineral density measurement in an identifiable and selectable manner before the radiation imaging.

18. An information processing method for an information processing apparatus that processes an image obtained by radiation imaging, the method comprising causing a display to display a thumbnail image display area that makes identifiable first imaging for bone mineral density measurement and second imaging different from the first imaging for the bone mineral density measurement before the radiation imaging.

19. A non transitory computer-readable storage medium storing a program configured to cause a computer to execute an information processing method defined in claim 17.
